(19) United States
(12) Patent Application Publication
(10) Pub. No.: US 2010/0055145 A1
(43) Pub. Date: Mar. 4, 2010

(54) STENT COATINGS FOR REDUCING LATE STENT THROMBOSIS

(75) Inventors: Ronald E. Betts, La Jolla, CA (US); Douglas R. Savage, Del Mar, CA (US); John E. Shulze, Rancho Santa Margarita, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Biosensors International Group, Hamilton (BM)

(21) Appl. No.: 12/201,969

(22) Filed: Aug. 29, 2008

(51) Int. Cl. A61F 2/04; A61P 9/00

(52) U.S. Cl. 424/423

(57) ABSTRACT

Devices and methods relate to drug-eluting stents and coatings, thereof, for reduced late stent thrombosis are described.

### TECHNICAL FIELD

The present devices and methods relate to drug-eluting stents and coatings, thereof, for reducing late stent thrombosis.

### BACKGROUND

First generation drug-eluting stents (DES) that provide for controlled release of sirolimus or paclitaxel from durable polymer stent coatings reduce angiographic and clinical restenosis compared to bare metal stents (BMS).¹⁻⁴ “Limus” drug analogues such as sirolimus and everolimus are more effective than paclitaxel to reduce neointimal growth and repeat revascularization procedures.⁵⁻⁹ However, restenosis still occurs, and very late stent thrombosis resulting from delayed healing, poor re-endothelialization, and other causes remains a potential problem for stent recipients.^{3,8,10} Thus, while current IDES stents are in many ways superior to BMS.^{3,4,8} they continue to carry an incremental risk of very late stent thrombosis.^{3,8,10,23} Although the mechanisms leading to very late stent thrombosis are poorly understood, the stent surface polymer coating that is used for controlled drug-release, and which remains as a permanent encapsulant of the implanted metal stent, has been implicated as a possible cause. The clinical consequences of stent thrombosis are generally catastrophic, including short-term mortality rates in the range of 20% to 25%; major myocardial infarction in 60% to 70% of cases; and six-month mortality rates, among survivors of stent thrombosis, in the range of 20% to 25%.

In a recent clinical study, patients who suffered a ST-elevation myocardial infarction (STEMI) due to stent thrombosis were more lIkely to have unsuccessful reperfusion, have a new myocardial infarction (MI), or die in-hospital compared to STEMI patients whose MIs were caused by de novo coronary artery disease.^{37,38} The Chechi et al. study compared clinical characteristics and outcomes in 115 patients with STEMI due to stent thrombosis with 98 patients with de novo STEMI, all of whom underwent PCI. Successful reperfusion rates were lower, while distal-embolization rates, in-hospital death rates, reinfarction and repeat target vessel revascularization (TVR), were higher in the stent-thrombosis group (Table 1). These findings underscore the increased risks associated with stent thrombosis, particularly important in light of the ongoing debate over the continuing long term stent-thrombosis risk which might be associated with drug-eluting stents (DES). Cumulative results obtained over six-months in the Chechi study identified statistically significant differences in rates of death, MI and stent thrombosis, all favoring the de novo STEMI group.

TABLE 1
Procedural and in-hospital results of the Chechi study

| | De novo | STEMI with stent | |
|---|---|---|---|
| Outcome | STEMI (%) | thrombosis (%) | P value |
| Successful reperfusion | 96.9 | 80.4 | 0.0001 |
| Distal embolization | 0.0 | 6.5 | 0.01 |
| Residual dissection | 1.0 | 16.3 | 0.0001 |
| Death | 7.1 | 17.4 | 0.03 |
| MI | 1.0 | 8.1 | 0.02 |
| TVR | 2.0 | 9.3 | 0.009 |

Another study compared the net benefit of DES versus BMS in terms of quality-adjusted life expectancy (QALE) and concluded that the small increase in very late stent thrombosis (VLST) with DES (0.14% over 4 years of follow-up) was sufficient to make the implantation of BMS the preferred strategy in the test PCI population (Table 2).³³

TABLE 2
Quality-Adjusted Life Years (QALYs) for Differing Thrombosis Risks

| Incremental Risk of | | | |
|---|---|---|---|
| VLST | DES QALYs | BMS QALYs | Difference in Risk |
| QALYs for DES | | | |
| Equal Risk | 16.262 | 16.248 | +0.014 |
| Risk Increase | | | |
| of 0.13% | | | |
| per Year for DES | 16.254 | 16.253 | +0.001 |

VLST = very late stent thrombosis occurring >1 year after coronary stent implantation

Thus, although DES reduce restenosis and target lesion revascularization compared with BMS, the increased risk of late stent thrombosis has curbed enthusiasm for the widespread use of DES. The need exists for DES that control restenosis and very late stent thrombosis, eliminating the need to balance risk factors when selecting a stent.

### REFERENCES

The following references, and additional references cited herein, are hereby incorporated by reference in their entirety.

1. Moses, J. W. et al. (2003) N. Engl. J. Med. 349:1315-23.

2. Stone, G. W. et al. (2004) N. Engl. J. Med. 350:221-31.

3. Stone, G. W. et al. (2007) N. Engl. J. Med. 356:998-1008.

4. Kastrati, A. (2007) N. Engl. J. Med. 356:1030-9.

5. Windecker, S. et al. (2005) N. Engl. J. Med. 353:653-62.

6. Kastrati, A. et al. (2005) JAMA 294:819-25.

7. Schomig, A. et al. (2007) J. Am. Coll. Cardiol. 50:1373-80.

8. Stettler, C. et al. (2007) Lancet. 370:937-48.

9. Stone, G. W. et al. (2008) JAMA 299:1903-13.

10. Bavry, A. A. et al. (2006) Am. J. Med. 119:11056-61.

11. Maisel, W. H. et al. (2007) N. Engl. J. Med. 356:981-4.

12. Grube, E. et al. (2006) Expert Rev. Med. Devices 3:731-41.

13. Grube, E. et al. (2005) EuroIntervention 1:53-57.

14. Kaiser, C. et al. (2005) Lancet 366:921-29.

15. Newcombe, R. G. et al. (1998) Stat. Med. 17:873-90.

16. Morice, M. C. et al. (2006) JAMA 295:895-904.

17. Kereiakes, D. et al. (2005) J. Am. Coll. Cardiol. 45:1206-12.

18. Krucoff, M. W. (2008) J. Am. Coll. Cardiol. 51:1543-52.

19. Laarman, G. J. et al. (2006) N. Engl. J. Med. 355:1105-13.

20. Spaulding, C. et al. (2006) N. Engl. J. Med. 355:1093-104.

21. Wiviott, S. D. (2007) N. Engl. J. Med. 357:2001-15.

22. Serruys, P. et al. (2006) EuroIntervention 2:286-94.

23. Daemen, J. et al. (2007) Lancet 369:667-78.

24. Beohar, N. et al. (2007) JAMA 297:1992-2000.

25. Win, H. K. et al. (2007) JAMA 297:2001-09.

26. Abizaid, A. et al. (2004) Am. J. Cardiol. 94:6.

27. Huang, S. et al. (2003) Cancer Biol. Ther. 2:222-32.

28. Kahan, B. D. (2001) Expert Opin. Pharmacother. 2:1903-17.

29. Kirchner. G. I. et al. (2004) Clin. Pharmacokinet. 43:83-95.

30. Kovarik, J. M. et al. (2003) Expert Opin. Emerg. Drugs 8:47-62.

31. Nashan, B. (2002) Expert Opin. Investig. Drugs 11:1845-57.

32. Sehgal, S. N. (2003) Transplant Proc. 35:7S-14S.

33. Garg, P. et al. (2008) J. Am. Col. Cardiol. 13:1844-53

34. Grube, E. et al. (2004) STEALTH-1@30 Days: Early Findings, Euro-PCR 04 Scientific Sessions, Paris, May 22-28.

35. Mehran, R. et al. (2004) Oral Abstract #3494: “First In Man Experience of the Biolimus A9 Drug Eluting Stent (MATRIX-Stent) in Treatment of Denovo Coronary Lesion: Results from the STEALTH-I Trial,” AHA Scientific Sessions, November 7-10.

36. Abizaid, A. et al. (2005) Oral Abstract: “Quantitative Angiographic Findings Demonstrate Equivalent Efficacy in Higher Risk Lesions Treated with Biosensors BioMATRIX™ Biolimus A9-Eluting Coronary Stent,” ACC '05, January 5.

37. Chechi, T. et al. (2008) J. Am. Coll. Cardiol. 51:2396-2402.

38. Alfonso, F. (2008) J. Am. Coll. Cardiol. 51:2403-05.

### SUMMARY

The following aspects and embodiments thereof described and illustrated below are meant to be exemplary and illustrative, not limiting in scope.

In one aspect, a method of stent-placement percutaneous coronary intervention (STPCI) that is effective to achieve a significant reduction in the extent of late restenosis, relative to that observed in a STPCI method in which a bare metal stent is placed at the site of vessel occlusion, without a concomitant increase in stent thrombosis up to 4 years following stent placement, is provided. The method comprises:

selecting as a subject for the method, an individual having a coronary occlusion condition characterized by a baseline stenosis >50%, a left main occlusion <50%, and a left ventricular ejection fraction (LVEF) of at least 30%, and

implanting at the site of the vessel occlusion, a drug-eluting stent having a metal body whose outer surfaces have been treated to enhance the adhesion of a biodegradable polymer coating and having on the treated outer surfaces, a drug/polymer coating formulated to contain at least 40% by weight of a macrocyclic triene anti-restenosis drug in a polylactic acid (PLA) polymer, at a coating thickness such that the both the drug and coating are completely released from the stent body over a period of between 6-12 months.

In some embodiments, the drug-polymer coating contains a 1:1 mixture by weight of drug and polymer, and the PLA polymer is D,L-PLA.

In some embodiments, the limus drug is selected from the group consisting of rapamycin, everolimus, zotarolimus, biolimus A9, novolimus, RAPALOG #AP23573. In particular embodiments, the limus drug is biolimus A9.

In some embodiments, the coating is applied in an amount equal to about 14-16 μg/mm stent length.

In some embodiments, the stent body outer surfaces have been treated by applying a coating of parylene to the surfaces by vapor deposition, for example, to enhance the adhesion of the biodegradable polymer coating.

In some embodiments, the stent body outer surfaces have been treated by plasma cleaning, for example, to enhance the adhesion of the biodegradable polymer coating.

In some embodiments, the matrix is coated on the exterior surfaces of the stent in an amount of about 15.6 μg/mm of stent length.

In some embodiments, the biodegradable polymer is polylactic acid (PLA). In particular embodiments, the biodegradable polymer is D,L PLA.

In a related aspect, a drug-eluting stent having a metal body whose outer surfaces have been treated to enhance the adhesion of a biodegradable polymer coating and having on the treated outer surfaces, a drug/polymer coating formulated to contain at least 40% by weight of a macrocyclic triene anti-restenosis drug in a polylactic acid (PLA) polymer, at a coating thickness such that both the drug and coating are completely released from the stent body over a period of between 6-12 months for use in treating a coronary occlusion in an individual having a coronary occlusion condition characterized by a baseline stenosis >50%, a left main occlusion <50%, and a left ventricular ejection fraction (LVEF) of at least 30%, by implanting the stent at the site of the coronary occlusion is provided, whereby implantation of the stent achieves a significant reduction in the extent of late restenosis, relative to that observed in a PCI method in which a bare metal stent is placed at the site of vessel occlusion, without a concomitant increase in stent thrombosis up to four years following stent placement.

In another related aspect, the use of a drug-eluting stent having a metal body whose outer surfaces have been treated to enhance the adhesion of a biodegradable polymer coating and having on the treated outer surfaces a drug/polymer coating formulated to contain at least 40% by weight of a macrocyclic triene anti-restenosis drug in a polylactic acid (PLA) polymer, at a coating thickness such that both the drug and coating are completely released from the stent body over a period of between 6-12 months for the preparation of a medicament for treating a coronary occlusion in an individual having a coronary occlusion condition characterized by a baseline stenosis >50%, a left main occlusion <50%, and a left ventricular ejection fraction (LVEF) of at least 30%, by implanting the stent at the site of the coronary occlusion is provided, whereby implantation of the stent achieves a significant reduction in the extent of late restenosis, relative to that observed in a PCI method in which a bare metal stent is placed at the site of vessel occlusion, without a concomitant increase in stent thrombosis up to four years following stent placement.

In addition to the exemplary aspects and embodiments described above, further aspects and embodiments will become apparent by reference to the drawings and by study of the following descriptions.

### BRIEF DESCRIPTION OF THE DRAWINGS

FIG. 1 illustrates an exemplary bare metal stent having a 6-crown pattern.

FIG. 2 illustrates an exemplary bare metal stent having a 9-crown pattern.

FIG. 3 shows the structure of the limus drug BIOLIMUS A9.

FIG. 4 illustrates a two-step process for synthesizing BIOLIMUS A9.

FIG. 5 illustrates a balloon catheter device for delivering a coronary stent.

FIG. 6 shows the dimensions of a particular balloon catheter device for delivering a coronary stent.

FIGS. 7A and 7B show tables summarizing in-hospital complications and out-of-hospital complications, respectively, in DES recipients up to 1,440 days following implantation.

FIG. 8 shows a graph comparing PLA degradation and BIOLIMUS A9 release.

FIG. 9 is a table showing the amount of recoil obtained using different types of stents.

### DETAILED DESCRIPTION

### I. Definitions

Prior to describing the present device and methods, the following terms are defined for clarity. Terms and abbreviations not defined should be accorded their ordinary meaning as used in the art.

As used herein, “deaths” are classified as cardiac or non-cardiac and/or procedure-related. Drug and device-related deaths may be further categorized by particular protocols.

As used herein, “cardiac death” is defined as any death that is not clearly attributable to a non-cardiac cause. Cardiac death includes but is not limited to death due to acute myocardial infarction (AMI), heart failure, congestive heart failure (CHF), cardiogenic shock, pulmonary edema, hypotension (systolic BP <80 mmHg), respiratory failure, cardiac perforation/pericardial tamponade, arrhythmia, bradycardia (heart block), cerebrovascular accident, non-cardiac complication of a cardiac procedure (including bleeding, vascular repair, transfusion reaction, or bypass surgery), unless other etiology is clearly responsible for the condition.

As used herein, “procedure-related deaths” refers to deaths directly related to a procedure or complications, thereof, or any death occurring within 30 days of a procedure.

As used herein, “myocardial Infarction” or “MI” refers to a condition that occurs when the blood supply to any part of the heart is interrupted. MI is broadly classified as Q wave or non-Q wave in etiology.

As used herein, “Q wave MI” is indicated by new pathologic Q waves in two or more contiguous EKG leads as determined by the EKG core laboratory or independent review of the CEC (Clinical Events Committee) and any elevation of cardiac enzymes and/or chest pain or other acute symptoms consistent with myocardial ischemia and new pathologic Q waves in two or more contiguous EKG leads as determined by the EKG core laboratory or independent review of the CEC, in absence of timely cardiac enzyme data. In the absence of ECG data, a Q wave MI condition may be identified using cardiac enzyme data along with other clinical data.

As used herein, “non-Q wave MI” is based on the Modified World Health Organization (WHO) definition (FDA MI; i.e., elevation of CK to more than two times normal with elevated CKMB) and/or the CDAC Classification (peri-procedural MI only), which recognizes the following classification of MI: Class III (Q waves present in two or more leads or CK-MB>8× normal, or if CK-MB data is not available then CK>3 times normal); Class II (CK-MB≧3 and ≦8× normal, or CK-MB>1 and <3× normal in the presence of major new EKG changes, or if CK-MB data are not available then CK>2 times normal): and Class I (CK-MB>1 and <3× normal, without major new EKG changes, or if CK-MB data are not available then CK>1 times normal qualified). Early or late non-Q wave MI may also be defined as CK-MB>3 times normal. Where cardiac enzyme elevations occur after CABG, non-Q wave MI is defined as CK>3 times normal or CK-MB>5 times normal. If CABG occurred after a procedure complication (e.g., emergent CABG), then the MI was also classified according to the CDAC classification scheme.

As used herein, “recurrent MI” is defined as re-elevation of CK-MB (or CK, if MB data not available) by more than 20% after more than a 20% decline from previous peak value.

As used herein, “late loss” and “very late loss” refer to the absolute value of increase in thickness of neointimal tissue within a previously treated coronary vessel over time. As used herein, late loss refers to a period specified between six months and one year, while very late loss refers to late loss after one year and up to about 4 years. As used herein, “repeat revascularization” refers to a revascularization procedure associated with a particular target lesion or target vessel which has previously undergone a revascularization procedure.

As used herein, “emergent revascularization” refers to revascularization associated with complications relating to the stent implantation procedure, including subacute closure of the target vessel in the first 24 hours following implantation.

As used herein, the term “clinically driven,” as it pertains to revascularization, refers to the clinical/medical necessity for repeat revascularization based on the presence of symptoms including ischemia (i.e., recurrent angina or equivalent) coupled with stenosis in excess of 50% of the diameter of the blood vessel or implanted stent, or in the absence of ischemia, stenosis in excess of 70% of the diameter of the blood vessel or implanted stent.

As used herein, target lesion revascularization (TLR) refers to repeat revascularizations that involve the originally treated vascular segment or a segment of the vasculature within about 5 mm of the stented segment. For example, a vessel treated at the site of a previously treated lesion in a coronary artery due to reocclusion of that previously treated lesion would be considered a TLR.

As used herein, “target vessels” include all coronary segments in the same epicardial artery as a treated lesion if the segments were involved in the passage of a coronary guidewire or any other device involved in stent implantation or other procedures.

As used herein, “abrupt and subacute closure” refer to the occurrence of reduced flow (TIMI grade 0 or 1) in a target vessel that persists and requires rescue by another revascularization device or by emergency surgery. “Abrupt closure” relates to a mechanical dissection (of the treatment site or other instrumented site), coronary thrombus or severe spasm, but does not connote “no reflow,” in which case the epicardial artery is patent but reduced flow persists, nor transient closure and reduced flow, in which case further randomized treatment reverses the closure. “Subacute closure” refers to abrupt closure that occurs after a stent implantation procedure is completed and the patient has left the catheterization laboratory. “Threatened closure” refers to any of the following conditions where there is no progression to frank abrupt closure: (i) dissection ≧NHLBI C, (ii) dissection NHLBI B and >50% diameter stenosis, (iii) diameter stenosis >70%, (iv) reduced flow (<TIMI 3) with residual >50% stenosis or any dissection, (v) symptoms of ischemia.

As used herein, “stent thrombosis” is generally defined as either an acute (<24 hours) or subacute (24 hours-30 days) condition associated with an occlusion at a site of stent implantation or death occurring within 30 days of stent implantation that is not explained by a cause other than stent occlusion.

As used herein, “late stent thrombosis” refers to thrombosis that occurred after 30 days and up to one year following implantation of a new stent. The HCRI CEC has proposed the following definitions for late stent thrombosis, which are adopted, herein:

Definite Late Stent Thrombosis:
Myocardial infarction that occurs >30 days after an implantation procedure and is attributable to the target vessel,
Angiographic documentation (site-reported or by quantitative coronary angiography [QCA]) of thrombus or total occlusion at the target site, and
In the absence of an interim revascularization of the target vessel.

Possible Late Stent Thrombosis:
Myocardial infarction that occurs >30 days after an implantation procedure and attributable to the target vessel
No identifiable “culprit” lesion elsewhere,
And in the absence of an interim percutaneous revascularization of the target lesion, and
In the absence of interim bypass grafting of the target vessel.

Special Situations:
A myocardial infarction that occurs immediately after a percutaneous or surgical revascularization procedure is not considered late stent thrombosis.
The prerequisite for definite or possible late stent thrombosis is clinical presentation consistent with an acute myocardial infarction attributable to the target vessel. If a patient undergoes primary angioplasty or thrombolytic therapy, no enzymatic or ECG criteria are required.
At the CEC meeting on Mar. 22, 2000, members voted to apply the above definitions to native vessels only. While it was appreciated that LST might also occur in vein grafts, it was believed impossible to differentiate between LST and SVG disease progression. When there is an acute event and total occlusion in vein grafts, the event is referred to as a total occlusion with MI (TOMI).

As used herein, “major vascular complications” refer to any vascular complication that requires surgical repair, ultrasound compression, or transfusion.

As used herein, “major bleeding” refers to bleeding that results in 25% or greater decline in hematocrit (HCT) (e.g., 30 to 40) or requires transfusion.

As used herein and in the appended claims, the singular forms “a”, “an”, and “the” include plural reference, unless the context clearly dictates otherwise.

Unless defined otherwise, all technical and scientific terms used herein have the same meanings as commonly understood by one of ordinary skill in the art to which this subject matter belongs. Although any methods and materials similar or equivalent to those described herein can be used in the practice or testing of the present subject matter, the preferred methods, devices, and materials are described below.

All publications mentioned herein are incorporated herein by reference for the purpose of describing and disclosing the methodologies which are reported in the publications which might be used in connection with the subject matter herein.

The present device, system, and method are now described with reference to the accompanying drawings and tables.

### II. Drug Eluting Stents (DES) for Reducing Late Restenosis

A. Introduction

The present device, system, and method relate to a drug fluting coronary stent (DES) having an exterior surface coated with a matrix comprising a biodegradable polymer and a limus drug. A feature of the device, system, and method is that drug release and polymer degradation are concurrent over a preselected period of time, typically 6-12 months, after which no polymer nor drug remain to adversely affect recovery. These and other features of the device, system, and method to reduce very late stent thrombosis without adversely affecting restenosis (i.e., neointimal formation after about four years of stent implantation) or other aspects of recovery, in subjects having a coronary artery lesion and a left ventricle ejection fraction (LVEF) of at least about 30%, are described in further detail below.

The DES and associated system and method are based on a balloon-expandable drug eluting stent that includes a stainless steel bare metal stent (i.e., the S-Stent™) having a primer coating or otherwise having undergone a surface preparation process such as plasma cleaning to improve adhesion of a polymer (infra) to its surface. The stent is coated with a biodegradable polymer coating containing a limus drug as an active pharmaceutical ingredient. The stent can be delivered using a rapid exchange delivery system, as herein described.

B. Metal Stent

The DES includes a metal endovascular stent upon which a drug/polymer matrix is coated. Endovascular stents are typically cylindrically-shaped devices capable of radial expansion. When placed in a body lumen, a stent in its expanded condition exerts a radial pressure on the lumen wall to counter any tendency of the lumen to close. Stents have found particular use in maintaining vessel patency following angioplasty, e.g., in preventing structural failure of the vessel and thus preventing interruption in blood flow through the vessel. In this application, a stent is inserted into a damaged vessel by mounting the stent on a balloon catheter and advancing the catheter to the desired location in the patient's body, inflating the balloon to expand the stent, and then deflating the balloon and removing the catheter.

The stent in its expanded condition in the vessel exerts a radial pressure on the vessel wall at the lesion site to counter any tendency of the vessel to close. “Self-expanding” stents are also known, made from spring material, mesh tubes, or shape-memory alloys. These devices are typically mounted on a catheter shaft surrounded by a sheath that constrains the expansion of the spring elements of the stent until the stent is positioned at the lesion site. Retraction of the sheath portion allows the stent to expand and contact the vessel lumen.

Numerous stent geometries and configurations are known in the art, and any of the geometries are suitable for use herein. The basic requirements of the stent geometry are (1) that it be expandable upon deployment at a vascular injury site, and (2) that it is suitable for receiving a coating of drug or for carrying a drug-containing coating on its surface, for delivering drug into the lumen in which the stent is placed. Preferably, the stent body also has a lattice or mesh structure, allowing viable endothelial cells in the stent “windows” to grow over and encapsulate the stent struts which are supporting the vessel lumen with living tissue. One skilled in the art will understand that there are numerous metal alloys that may be used for construction of a successful endovascular stent, including cobalt chromium (MP34a, L605: or F562) or nitinol, inconel, molybdenum and stainless steel. The alloy used may be further modified through the use of additives or through chemical or heat treatment processes to modify performance characteristics such as yield strength, ductility, flexibility, fracture resistance, scaffolding strength and radioopacity. Examples of materials that may be added to stents, either in a multiple layer sandwich format, by surface bombardment, or in a solid solution, while maintaining a relatively thin flexible stent structure to increase radioopacity, are tantalum, platinum or iridium. Stents may be created by laser cutting or by selective chemical etching of seamless or non-seamless hypotube using known laser cutting or photolithography techniques, or by chemical or vapor deposition plating over a stent pattern created on a sacrificial substrate.

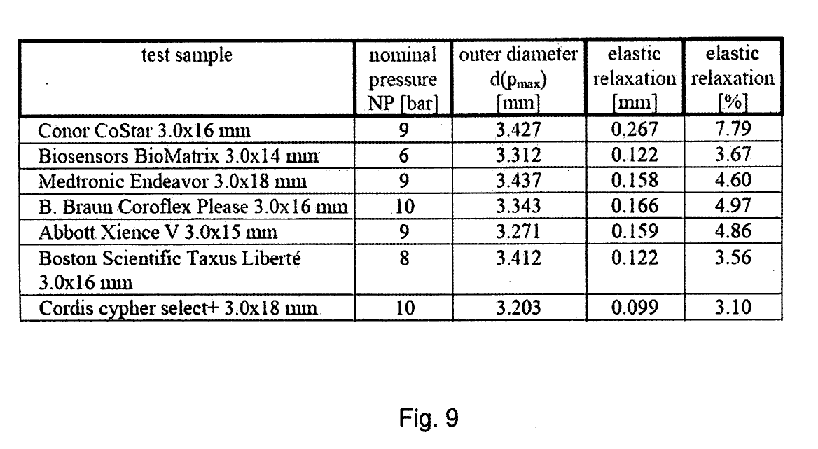

An exemplary stent is an endovascular stent which exhibits a low recoil after expansion of approximately 3% or less (FIG. 9). Many cobalt chromium alloy stents of the current art exhibit a recoil of 5% or more. Animal test data has shown that vascular injury increases directly with the degree of overexpansion of the stent relative to vessel reference diameter during the stent deployment and implantation procedure. Higher vascular injury is associated with higher inflammation during stent healing and higher restenosis. To achieve the same final desired stent diameter after implant, a stent with ˜3% or less recoil requires less overexpansion during the stent implant procedure and thus produces less vascular injury. An exemplary stent is a cobalt chromium alloy stent or a stainless steel stent with ˜3% or lower recoil. in one preferred embodiment the S-Stent™ (Biosensors International), which is laser cut from medical grade 316L stainless steel, and which complies with the requirements set forth in ASTM F138-03. The S-Stent™ is available in two patterns to accommodate a wide range of expansion diameters. These patterns are differentiated by the number of crowns (either 6 or 9). Each pattern includes a series of corrugated rings aligned along a common longitudinal axis. Each ring is connected to an adjacent ring by two or three (6 crown and 9 crown, respectively) connectors (also known as links), which are oriented in the direction of the longitudinal axis of the stent.

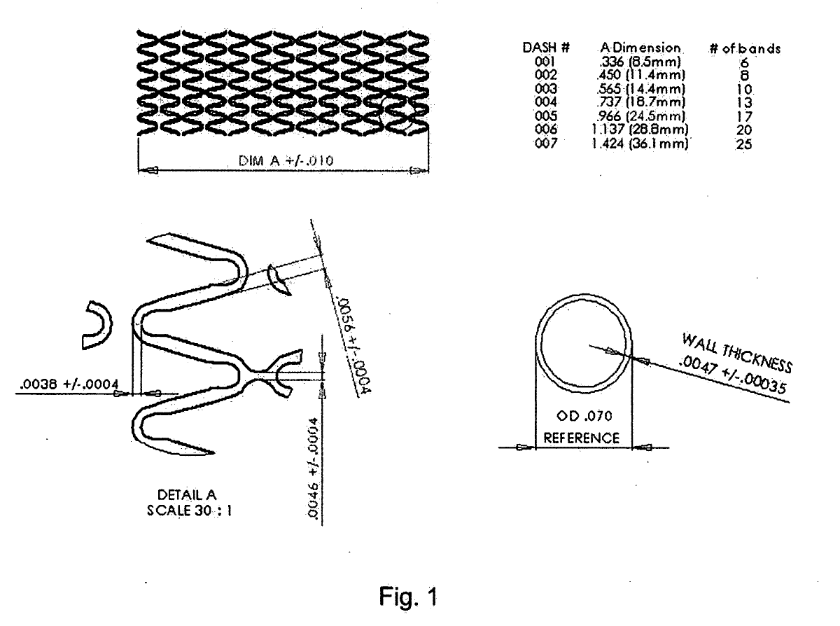

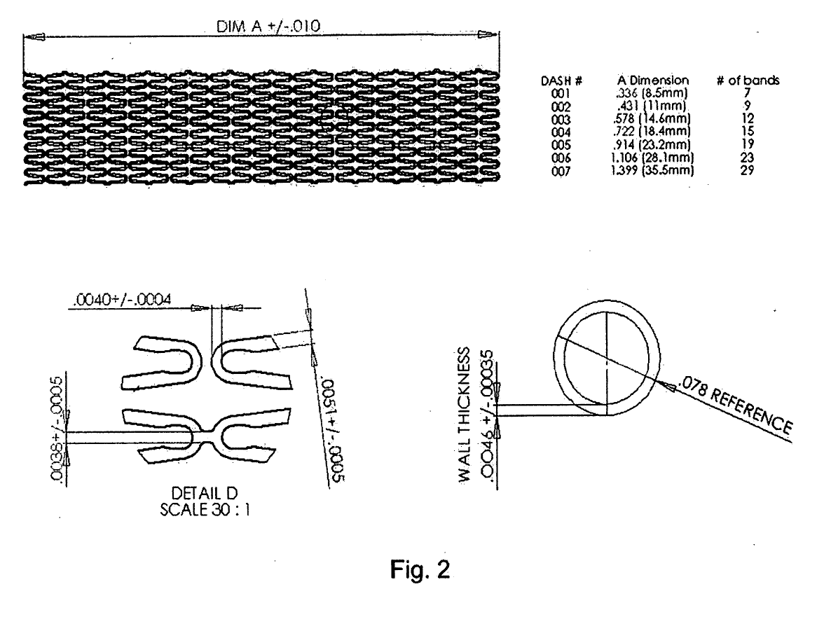

In the 6-crown pattern, the links are offset by 90 degrees about the stent circumference between successive corrugated rings (FIG. 1). In the 9-crown pattern, there is a 60 degree axial offset of the connectors in successive bands. (FIG. 2) Based upon the degree of offset of the connectors in adjacent bands, the peaks of the serpentine bands attached to the connector move axially during expansion such that the overall length of the stent is maintained based on the slight axial distortion of each successive ring. The 6-crown pattern is employed for 2.5 mm and 3.0 mm diameter stents and the 9-crown pattern is employed for 3.5 mm and 4.0 mm diameter stents The stents may be electropolished to obtain a smooth finish with a thin chromium dioxide surface layer, and then annealed to obtain preselected ductility, fatigue, and tensile characteristics.

The S-Stent™ was used as a control BMS in the FUTURE I (n=15), FUTURE II (n=43), and STEALTH FIM (n=40) studies, and in two separate registry trials in Asia (for a total of 225 patients). The 30 day MACE and long-term (6 and 12 month) results from these studies demonstrated that the bare metal S-Stent™ is safe and achieves similar or improved results when compared to commercially available stainless steel BMS. A summary of these data is provided, below.

C. Compositions and Methods For Enhancing Adhesion

In some embodiments, the stent is coated with a polymer underlayer composition to promote the adhesion of a subsequently applied drug/polymer matrix. Suitable polymers for forming polymer underlayers include but are not limited to poly(D, L-lactic acid), poly(L-lactic acid), poly(D-lactic acid), ethylene vinyl alcohol (EVOH), ε-caprolactone, ethylvinyl hydroxylated acetate (EVA), polyvinyl alcohol (PVA), polyethylene oxides (PEO), PARY-LAST™, PARYLENE (i.e., poly(dichloro-para-xylylene)), silicone, polytetrafluoroethylene (TEFLON®) and other fluoropolymers, and co-polymers thereof and mixtures thereof. The underlayer can be deposited from a solvent-based solution, by plasma-coating, vapour deposition, or by other coating or deposition processes (see, e.g., U.S. Pat. No. 6299,604). The underlayer typically has a thickness of between about 1 micron and 5 microns.

An exemplary polymer underlayer is formed from a para-xylylene polymer, which has been previously used to coat various implantable and short term exposure medical devices including stents, needles, mandrels, catheters, cardiac assist devices and prosthetics. Addition of the parylene coating in these applications enhances lubricity and corrosion resistance. A particular para-xylylene polymer, PARYLENE C, is a polymeric form of para-chloro-xylylene has been found to enhance the adhesion of a drug/biodegradable polymer layer to the stent ablumenal surface. PARYLENE C is chemically, biologically, and thermally stable, insoluble in organic solvents up to 150° C., and does not appear to degrade substantially in the body. PARYLENE C exhibits low permeability to moisture, chemicals and other corrosive gases. The chemical structure of PARYLENE C is shown below;

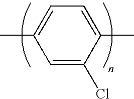

PARYLENE C can be applied to a stent using a vapor deposition polymerization process in which the dimer is first vaporized in a vacuum environment and then pyrolized to form a monomer. The monomer is then precipitated onto a cooler stent metal substrate surface under vacuum. The vapor deposition polymerization process affords a uniform coverage of parylene across the stent substrate including corners edges and crevices with a resulting clear, transparent polymer film on the surface of the stent. It has been discovered that this layer may serve as a primer layer for attaching the biodegradable PLA polymer. Exemplary thicknesses for the coating of PARYLENE C are in the range 2-5 μm. The coating process may be performed by, e.g., Specialty Coating Systems (SCS, Indianapolis, Ind. USA) or Advanced Coating (Rancho Cucamonga, Calif. USA).

Alternately, PARYLENE C can be combined with a second common type of para-xylylene polymer, i.e., PARYLENE N, to form a primer layer. PARYLENE N exhibits a slightly lower tensile and dialectric strength than PARYLENE C, but is otherwise similar in chemical properties, and may be combined with PARYLENE C during vapor phase deposition. The chemical structure of PARYLENE N is C₈H₈ or poly (4-xylylene) whereas the chemical structure of PARYLENE C is C₈H₇Cl or monochlorinated poly (4-xylylene). In one example, the PARYLENE C/PARYLENE N coating consists of 14% PARYLENE C and 86% PARYLENE N. Tests have confirmed equivalent adhesion of PLA polymers (see below) to such mixtures.

In other embodiments, the adhesion of a drug/polymer is enhanced by cleaning and activating of the metal stent surface prior to drug/polymer coating. In one example, cleaning and activation was performed by exposure of the S-Stent to an argon or hydrogen plasma, and then by subsequently applying the drug/polymer directly to the surface of the stent.

D. Polymer/Drug Matrix

After coating with a material to enhance adhesion and/or cleaning and activation of the metal stent a polymer/drug matrix is applied to the stent surface. A polymer/drug matrix can be applied to all surfaces of the stent or only a preselected surface, such as the exterior surface. In an exemplary embodiment, the drug/polymer was applied in the form of an acetone-solvent based mixture of Biolimus drug and D,L-PLA polymer. Other suitable solvents for forming coating mixtures of the limus drugs and PLA polymers include ethylene acetate, chloroform, and methylene chloride.

1. Polymer

Preferred polymers for use in forming the polymer/drug matrix are polyesters of lactic acid known as polylactic acids (PLAs) or polylactides. PLAs are commonly synthesized by a method involving ring opening polymerization of a cyclic lactic acid dimer, i.e., a lactide (below), although it is possible to synthesize PLA by direct polycondensation.

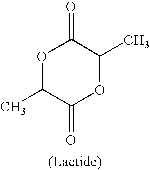

Lactide dimers are chiral molecules that exist in two stereoisomeric forms, i.e., D and L, which form either D-PLA or L-PLA. A racemic form of the polymer, i.e., D, L-PLA, can also be obtained. The repeating unit in a PLA molecule is generally represented by the following structure, where n is the degree of polymerization:

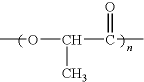

PLA derived from these optically active D and L monomers is a semicrystalline material. The particular physical properties of a PLA depend on its molecular weight and crystallinity. In some embodiments, D,L-PLA containing randomly distributed blocks of D-lactic acid and L-lactic acid along polymer chains may be used. In an exemplary embodiment, the particular PLA composition produces a polymer having a predominantly amorphous structure, rather than a crystalline structure, which provides more uniform biodegradation. Varying the ratio of D and L blocks in the polymer “fine tunes” the polymer for a particular application. Generally, the degree of crystallinity of the polymer varies with its stereoregularity, and is an important factor in biodegradation.

PLAs are commonly used as biomaterials for wound closure, prosthetic implants, and drug delivery systems. The monomer selection affords manufacturers the ability to control the rate of degradation which occurs by hydrolysis. Upon degradation, PLAs release non toxic lactic acid which is further converted into water and carbon dioxide via the Krebbs Cycle.

A comparison of typical thermal and mechanical properties of various commercially available PLAs is shown in Table 3, where MW refers to molecular weight (Dalton), Tg refers to glass transition temperature, and Tm refers to melting temperature of crystalline materials. The shaded values closely represent the D, L-PLA used for the clinical studies to be described.

TABLE 3
Thermal and Mechanical Properties of PLAs

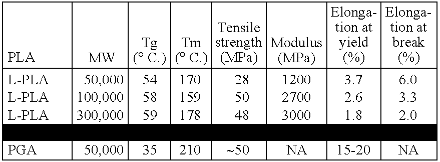

An exemplary D,L-PLA used in the clinical study to be described has a molecular weight of 75K-115K Dattons and a viscosity of 0.55-0.75 dL/g. The glass transition temperature (Tg) is ˜50-60° C. Table 4 lists the material properties of this D, L-PLA.

TABLE 4
Material Properties of the D, L-PLA used in the BIOMATRIX ® Stent

| Material Property | Characteristics/Ranges |
|---|---|
| Appearance | White to faintly yellow; Granular to pelletized solid |
| Identification | Match reference spectrum |
| H-NMR | |
| Inherent Viscosity | 0.60-0.70 dL/g in chloroform (CHCl₃) at 30° C. |
| Residual tin (Sn²⁺) | 200 ppm (max.) |
| Bioburden (Aerobic, | 100 total colony forming units (CFUs)/gram |
| Aerobic Spores, | |
| Anaerobic) | |
| Pyrogenicity, LAL | 0.5 EU/mL (max) |
| Residual Monomer | <2 mole % |
| Residual Toluene | <890 ppm |

In the particular stents described herein, para-xylylene polymer coated or plasma cleaned and activated metal S-stents were coated with a 1:1 mixture (wt/wt) of a polymer/drug matrix comprising polylactic acid.

2. Drug

The drug is preferably a macrolide triene lactone or “limus” drug, such as rapamycin or a derivative, thereof. Such derivatives include but are not limited to sirolimus, everolimus, zotarolimus, novolimus, tacrolimus, ABT 578 (Abbott Pharmaceuticals), Rapalog #AP23573 (Ariad Pharmaceuticals), BIOLIMUS A9 (Biosensors International), and another chemically related compounds that possess anti-cell proliferative, anti-restenotic, and/or anti-thrombotic activities.

An exemplary limus drug for use in preparing a drug/polymer matrix is BIOLIMUS A9,CAS-851536-75-9, which may herein be referred to as “biolimus,” the “biolimus drug,” or “BA-9.”

BIOLIMUS A9 is a semi-synthetic macrolide triene lactone and rapamycin 42-O-alkoxyalkyl derivative containing the characteristic 31-membered ring that is also present in sirolimus, everolimus, zotarolimus, novolimus, and Ariad Pharmaceuticals'Rapalog #AP23573. In particular, BIOLIMUS A9 is a highly lipophilic, semi-synthetic sirolimus analogue with an alkoxy-alkyl group replacing hydrogen at position 42-O.¹² BIOLIMUS A9 is approximately ten times more lipophilic than sirolimus or everolimus, with a partition coefficient (log P) estimated at 7.63 (pH 7.40). BIOLIMUS A9 was specifically developed for in vivo release from coronary stents to prevent smooth muscle cell proliferation.

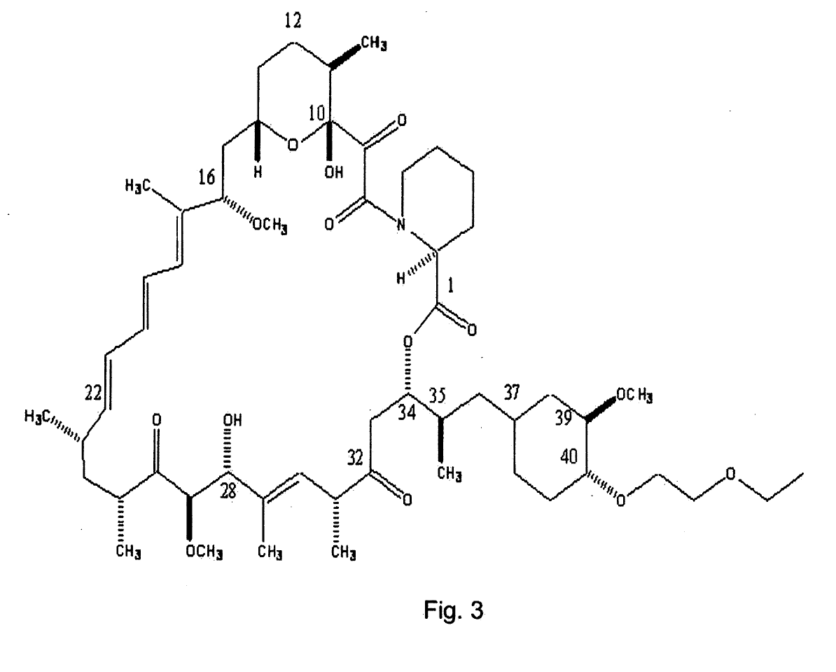

The chemical structure of BIOLIMUS A9 is depicted in FIG. 3. A summary of physical and chemical properties is provided in Table 5. The drug is described in detail in, e.g., U.S. Pat. No. 7,220,755, which is herein incorporated by reference.

TABLE 5
Physical-Chemical Properties of BIOLIMUS A9

| | |
|---|---|
| Appearance | White powder |
| Molecular Weight | 986.29 |
| Molecular Formula | C₅₅H₈₇NO₁₄ |
| Partition Coefficient (E Log P_{oct}): As | Log P_{n-octanol} = 7.63 |
| measured by reverse phase HPLC | |
| Stereochemistry | Two each interconvertible |
| | conformational isomers and |
| | tautomers |
| Crystalline Forms | Amorphous |
| Solubility: | |
| methanol | Very soluble |
| ethanol | Very soluble |
| ethyl acetate | Very soluble |
| water (independent of pH) | Practically insoluble |
| 25% ethanol in water | Practically insoluble |

Like sirolimus and everolimus, BIOLIMUS A9 is rapidly absorbed in tissues and is able to reversibly bind to immunophilins (cytoplasmic proteins) found inside living cells. Based upon the ubiquitous rapamycin ring structure present in the BIOLIMUS A9 molecule, it is believed that BA9 forms a complex with intracellular FKBP-12, which binds to the mammalian target of rapamycin (mTOR) to reversibly inhibit cell cycle transition of proliferating smooth muscle cells from the G₁ to S phase. BIOLIMUS A9 has been shown to inhibit the growth of proliferating animal and human smooth muscle cells in culture with a potency similar to that of sirolimus.

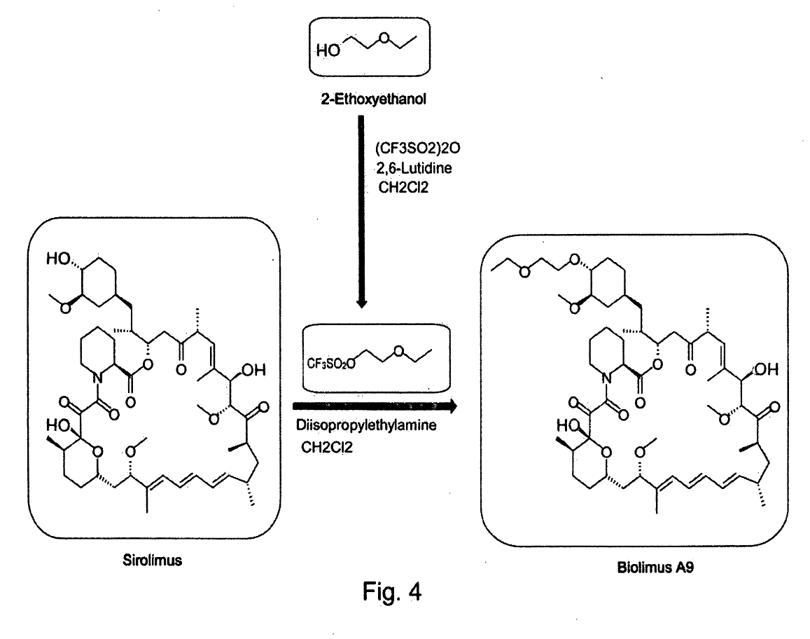

BIOLIMUS A9 may be synthesized via a two-step process as diagrammed in FIG. 4 and detailed in Example 3. In the first step of the process, 2-ethoxyethanol is modified to an intermediate, 2-ethoxyethanol triflate, via reaction with trifluoromethanesulfonic anhydride, and 2,6-lutidine in dichloromethane. The resulting intermediate, 2-ethoxyethanol triflate, is then purified by distillation and combined with commercially obtained rapamycin, diisopropylethylamine, and dichloromethane to yield BIOLIMUS A9, which can be purified by chromatography and solidified under evaporation from a methanol-water mixture.

For coating a BIOLIMUS A9/polymer matrix onto a stent, a polymer, such as D,L-PLA, is dissolved in a suitable solvent, such as acetone, and BIOLIMUS A9 is added to produce a drug/polymer matrix in solvent for coating onto a stent surface. In some embodiments, approximately equal weights of PLA and BIOLIMUS A9 (i.e., 1:1 wt/wt) are used to produce the matrix. The surface of the stent may have been previously coated with an adhesion composition, such as a para-xylylene polymer, or cleaned and activated, such as by argon or hydrogen plasma. Following application to a stent and evaporation of the solvent, the thin BIOLIMUS A9/D, L-PLA polymer coating may have a weight after drying of about 15.6 μg/mm of stent length, although other coating amounts may produce acceptable results. The thickness of the drug/polymer layer is preferably from about 3 microns to about 30 microns.

The polymer regulates the release of the limus into surrounding tissues, and the polymer is co-released along with the drug over a roughly equal time frame of 9 months. The limus drug/polymer matrix is preferably applied only to the ablumenal surfaces of a stent to reduce the release of the antiproliferative drug to the inside lumen of the stent where it would act to inhibit healing and thus reduce endothelial cell growth. On the ablumenal surface limus drug and polymer are co-released and co-absorbed with the polymer completely degrading to carbon dioxide and water during a period of about 6-9 months.¹²

### III Stent Delivery System

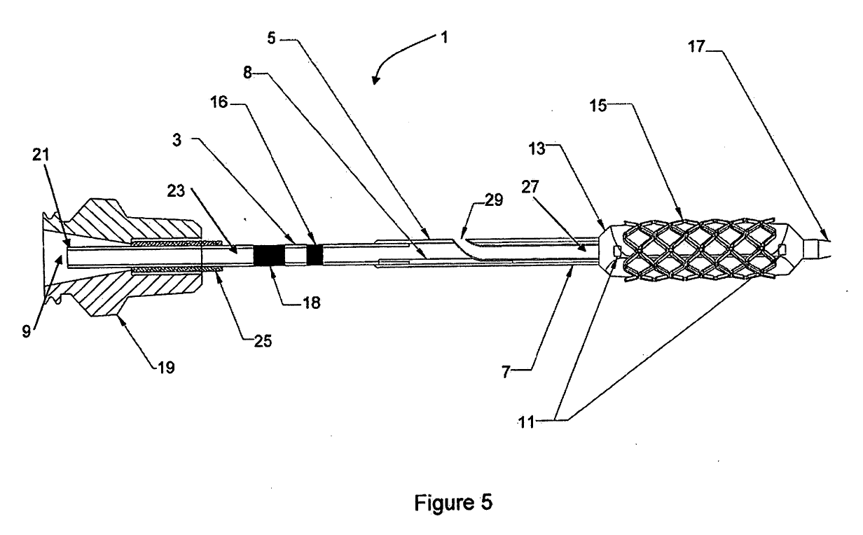

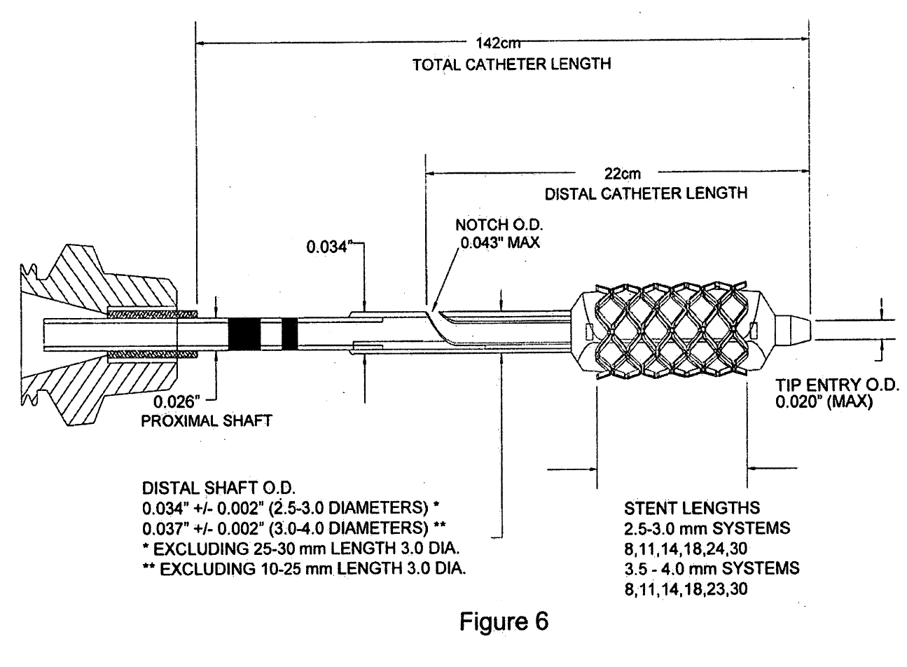

DES in accordance with the present devices, systems, and methods, are not required to be delivered to the site of a coronary artery lesion by any particular method or using any particular apparatus. The particular BIOMATRIX® DES used in the following clinical studies were crimped onto the distal balloon of a rapid exchange delivery system catheter 1 that combines a single lumen proximal shaft 3 with a dual lumen mid-shaft 5 and a coaxial lumen distal shaft 7 to create rapid exchange capability (FIG. 5). The catheter 1 may include a core wire 8 to impart a desired amount of flexibility. The proximal shaft 3 and other components of the system 1 may be coated with a lubricating polymer such as polytetrafluoroethylene (PTFE; TEFLON®) and/or optionally with a hydrophilic polymer coating. The single lumen proximal shaft 3 connects the distal shaft 7 with the inflation port 9 of the catheter. Such a catheter system is known as the BIOMATRIX DELIVERY SYSTEM or the SENSO DELIVERY SYSTEM. In the particular embodiment of the catheter system 1 shown in FIG. 6, the overall length of the delivery system catheter 1 is 150 cm.

Radiopaque balloon markers 11 are located on the distal shaft 7 to indicate the length of the balloon 13. A stent 15 is mounted such that the markers 11 reflect the expanded stent 15 length. The radiopaque markers 11 aid in the fluoroscopic positioning of the stent 15 and in accurately positioning the catheter 1 for post-deployment dilatation, if necessary. In a particular embodiment, two markers 11 are located 90 cm and 100 from the distal tip 17, indicating when the distal tip 17 of the catheter 1 exits the tip of a brachial or femoral guiding catheter (not shown). Additional markers, such as a radial marker 16 and a brachial marker 18, may be present elsewhere on the catheter, e.g., on the proximal shaft 3.

A single arm adapter 19 is attached to the proximal end 21 of the catheter 1 and communicates with the inflation/deflation lumen 23. The proximal shaft 3 is bonded to the single arm adapter 19 using adhesives and an incorporated strain relief 25. Preferably, a 0.014-inch or smaller diameter guide wire (not shown) is used in the guide wire lumen 27. The guide wire exits the guide wire lumen 27 proximally at the guide wire exit notch 29, which is formed in the mid-shaft section 5. Proximal to this point, the guide wire runs external to and alongside the proximal shaft 3 of the catheter 1.

Optionally, the mid-shaft 5 and distal shaft 7 (including the distal tip 17 of the catheter 1) are coated with a hydrophilic coating. The coating is not applied to the working length of the balloon 13 or directly onto the stent 15. The purpose of the hydrophilic coating is to reduce the coefficient of friction of the catheter 1 and to aid in the advancement of the catheter 1 through the guiding catheter and the coronary anatomy.

FIG. 6 shows a particular catheter 1 for use as described, along with various physical dimensions. However, this particular catheter 1 described is only one example of a suitable delivery system for use with the present DES. Others are known in the art. The drawings shown in FIGS. 5 and 6 are not to scale.

### IV. Clinical and Experimental Data

The following clinical and experimental data support the present device, system, and method; however, the particular DES and method of implantation should not be construed as limiting.

A. Clinical Data

A previous Biosensors International study performed in humans (referred to as the “FUTURE I” study) evaluated the safety and performance of a Biosensors International DES (i.e., the “CHALLENGE” stent) compared to a non-eluting BMS (i.e., the S-Stent™). The CHALLENGE stent included a drug/polymer coating of another limus, i.e., everolimus/PLA.

In the FUTURE I study, a group of 36 patients with de novo coronary lesions (<18 mm length, between 2.75 and 4.0 mm reference diameter, and between ≧50% and ≦99% diameter stenosis) were randomized (2:1) to receive either the CHALLENGE DES stent (n=24) or the S-Stent™ as an uncoated control BMS (n=12). Angiographic and intravascular ultrasound evaluations were performed immediately after stent implantation/placement and again at 6 months and one year following implantation. The primary objective of the study was to demonstrate the safety of the CHALLENGE stent, as defined by the absence of 30-day major adverse clinical events (MACE) and freedom from restenosis as demonstrated by a reduction in late loss. The adjudicated results of the FUTURE I study demonstrated that the CHALLENGE DES stent produced an 80% reduction in late loss at 6 month angiographic follow-up, and only a 4% restenosis rate, compared to 8.3% for the control BMS.

Biosensors International subsequently developed a stainless steel stent (S-Stent™) coated with poly-lactic acid (PLA) and BIOLIMUS (i.e., the BIOMATRIX® drug eluting coronary stent system) to avoid neointimal thickening and restenosis. The present study, entitled the STEALTH trial (i.e., STent Eluting A9 bioLimus Trial in Humans) was performed to evaluate the safety and performance of this DES compared to a non-drug-eluting BMS (i.e., the same S-Stent™ used in the CHALLENGE study).³⁴⁻³⁶

The format and protocol of the STEALTH study are described below and in Examples 1 and 2. Briefly, a group of 120-patients were randomized 2:1 in a double-blinded fashion to be implanted with either the BIOMATRIX® DES or the S-Stent™ BMS (bare metal stent). Eighty patients were randomly assigned to receive the DES with BIOLIMUS A9 as the active drugs while forty patients received the control BMS. The study was conducted at Siegburg Heart Center and Brüderkrankenhaus Trier (two German heart centers), and at the Institute Dante Pazzanese of Cardiology (a medical research hospital located in São Paulo, Brazil).

Overall, BIOMATRIX® DES performed well in terms of safety and efficacy. MACE at six months was not statistically different between the DES and the BMS (3.8% for the DES, 2.5% for the BMS). However, the BIOLIMUS A9 eluting DES were much more effective than BMS in reducing in-stent late lumen loss (LL) (0.26 mm vs. 0.74 mm, p=<0.001) and binary restenosis (3.9% vs. 7.7%, p=0.4). The following description and tables provide an overview for the STEALTH trial protocol and patient population, and a detailed analysis of the data.

The protocol used in the STEALTH trial is summarized in Table 6, while a breakdown of patient demographics is shown in Table 7. The site investigator was responsible for screening potential patients, and collecting information from successful candidates prior to device stent implantation. Completion of patient history and provision of a fully executed patient informed consent were required prior to device implant. Cardiovascular risk factors identified in the patient population are shown in Table 8. Information relating to the baseline lesion characteristics in the study population is shown in Table 10. Randomization, at a 2:1 ratio (DES:BMS), occurred after initial angiographic characterization of target lesion(s). One implant failure resulted in a total of 119 patients eligible for continued study follow-up. A total of one-hundred nineteen stents were implanted.

TABLE 6
STEALTH Protocol Summary

| | |
|---|---|
| Study Title | STEALTH (STent Eluting A9 bioLimus Trial in Humans) |
| Study Design | A prospective, randomized, single-blinded, descriptive |
| | multi-centre clinical study to evaluate safety, tolerability, |
| | and performance of the drug eluting, BioMatrix Stent |
| Study Site Locations | Heart Centre Siegburg (Siegburg, Germany) |
| | Medizinische Klinik III (Trier, Germany) |
| | Instituto Dante Pazzanese de Cardiologia (São Paulo, Brazil) |
| Objective | To demonstrate the safety and efficacy of the BIOLIMUS |
| | A9 coated stent to reduce in-stent restenosis |
| Study Endpoints | |
| Primary Endpoint | Angiographic quantification of late loss as reduction in |
| | neointimal growth compared to bare metallic S-Stent ™ |
| Secondary Endpoints | Angiographic in-stent restenosis (>50%) at six-month |
| | follow-up, MACE, six-month IVUS results, and |
| | BIOLIMUS A9 levels in peripheral blood |
| Key Inclusion Criteria | Age ≧ 18 |
| | De novo lesions in native coronary vessels |
| | Baseline stenosis ≧50% |
| | Lesion length ≦24 mm |
| | Vessel reference diameter: (≧2.75 mm & ≦4.0 mm) |
| | No direct stenting |
| Key Exclusion Criteria | LVEF < 30% |
| | Left main occlusion ≧50% |
| | Chronic total occlusion, poor distal flow, thrombus |
| | Multiple stents (>2) implantation |
| | <7-day post AMI |
| | Side branch >2 mm |
| | Coexisting CHD, VHD, CRF |
| | Emergent procedure |
| Patient Enrollment: | |
| Initial enrollment | September 2003 |
| Enrolment complete | March 2004 |
| Patient follow-up visits | |
| Six-month | September 2004 |

TABLE 7
Patient Demographics

| | Control BMS | BIOLIMUS A9 | p-Value |
|---|---|---|---|
| Age (years) | 61.0 ± 9.2 | 62.0 ± 10.0 | 0.61 |
| Gender (Male) | 82.5% | 58.8% | 0.01 |

TABLE 8
Cardiovascular risk factors

| | Control BMS | BIOLIMUS A9 | p-Value |
|---|---|---|---|
| Diabetes Mellitus | 22.5% | 26.6% | 0.66 |
| Hypertension | 85.0% | 83.8% | >0.99 |
| Smoking | 61.5% | 46.3% | 0.12 |
| Family history of CAD | 30.6% | 50.7% | 0.06 |
| History of MI | 35.0% | 37.5% | 0.84 |
| Prior PTCA | 12.5% | 25.0% | 0.15% |
| Prior CABG | 2.5% | 10.0% | 0.27 |
| LV Ejection fraction | 60.2 ± 13.1% | 62.4 ± 11.1% | 0.61 |

TABLE 9
Baseline lesion characteristics

| Location | Control BMS | BIOLIMUS A9 | p-Value |
|---|---|---|---|
| Vessel | | | |
| LAD | 42.5% | 28.0% | 0.15 |
| LCX | 30.0% | 37.8% | 0.43 |
| RCA | 27.5% | 34.1% | 0.54 |
| Lesion | | | |
| Proximal | 42.5% | 48.8% | 0.57 |
| Mid | 47.5% | 41.5% | 0.56 |
| Distal | 10.0% | 7.30% | 0.73 |
| Ostial | 0% | 2.40% | >0.99 |

A summary of the acute quantitative angiographic findings reported for the enrolled patient population is shown in Table 10.

TABLE 10
Acute quantitative angiographic findings

| | Control BMS | BIOLIMUS A9 | p-Value |
|---|---|---|---|
| Reference vessel diameter | 2.97 ± 0.42 | 2.95 ± 0.40 | 0.79 |
| (mm) | | | |
| Lesion length (mm) | 13.75 ± 3.77 | 15.37 ± 4.64 | 0.06 |
| Pre-Procedure | | | |
| In-lesion MLD (mm) | 1.07 ± 0.28 | 1.02 ± 0.27 | 0.30 |
| In-lesion DS (%) | 64.07 ± 7.72 | 65.50 ± 7.76 | 0.34 |
| Post-Procedure | | | |
| In-stent MLD (mm) | 2.92 ± 0.33 | 2.89 ± 0.37 | 0.68 |
| In-lesion MLD (mm) | 2.48 ± 0.41 | 2.48 ± 0.38 | 0.93 |
| In-stent DS (%) | 2.62 ± 8.86 | 4.61 ± 9.70 | 0.28 |
| In-lesion DS (%) | 18.08 ± 8.02 | 18.64 ± 7.74 | 0.71 |

Table 11 summarizes the angiographic findings obtained at the six-month follow-up visit following implantation. Except for in-lesion and in-stent binary restenosis, values provided below are expressed as mean±standard deviation.

TABLE 11
Six-month quantitative angiography findings

| | Control BMS | BIOLIMUS A9 | p-Value |
|---|---|---|---|
| Reference vessel | 2.99 ± 0.43 | 3.00 ± 0.37 | 0.82 |
| diameter (RVD, mm) | | | |
| In-lesion MLD diameter | 2.08 ± 0.51 | 2.34 ± 0.45 | 0.01 |
| (mm) | | | |
| In-lesion DS (%) | 30.85% ± 11.91% | 22.03% ± 12.05% | <0.001 |
| In-stent MLD (mm) | 2.18 ± 0.56 | 2.64 ± 0.270 | <0.001 |
| In-stent DS (%) | 27.38 ± 13.92 | 11.86 ± 14.55 | <0.001 |
| In-lesion acute gain | 1.41 ± 0.38 | 1.47 ± 0.41 | 0.43 |
| In-lesion late loss | 0.40 ± 0.41 | 0.14 ± 0.45 | 0.004 |
| In-stent late loss | 0.74 ± 0.45 | 0.26 ± 0.43 | <0.001 |
| Proximal edge late loss | 0.17 ± 0.32 | 0.10 ± 0.25 | 0.23 |
| Distal edge late loss | 0.10 ± 036 | 0.08 ± 0.28 | 0.73 |
| In-lesion binary | 7.7% | 3.9% | 0.40 |
| restenosis | | | |
| In-stent binary restenosis | 7.7% | 3.9% | 0.40 |

Major adverse cardiac events (MACE) defined as a composite of death, elevation of CK-MB≧3 times upper limit of normal (sub-classified as Q-wave or non-Q-wave myocardial infarction) and target lesion revascularization (TLR) at one month and six months following stent implantation are presented, below, in a non-hierarchical (Tables 12 and 14) and hierarchical (Tables 13 and 15) manner. One patient (*) experienced spiral dissection during stent implant and received three non-study stents. One patient (**) had acute stent thrombosis and clinically driven re-PCI with no MI reported.

TABLE 12
MACE at one month (non-hierarchical)

| | Control BMS | BIOLIMUS A9 | p-Value |
|---|---|---|---|
| MACE (death, MI, or TLR) | 2.5% (1/40) | 3.8% (3/80) | >0.99 |
| Death | 0 | 0 | — |
| Myocardial Infarction | 2.5% (1/40) | 2.5% (2/80) | >0.99 |
| Q wave | 0 | 0 | — |
| Non-Q wave | 2.5%(1/40) | 2.5% (2/80) | >0.99 |
| Emergent CABG | 0 | 0 | — |
| Target lesion revascularization | 0 | 1.3% (1/80) | >0.99 |
| TL-CABG | 0 | 0 | — |
| TL-PTCA | 0 | 1.3% (1/80) | >.99 |

TABLE 13
MACE at one month (hierarchical)

| | Control BMS | BIOLIMUS A9 | p-Value |
|---|---|---|---|
| MACE (death, MI, or TLR) | 2.5% (1/40) | 3.8% (3/80) | >0.99 |
| Death | 0 | 0 | — |
| Q-wave MI without death | 0 | 0 | — |
| Non-Q wave MI without | 2.5% (1/40) | 2.5% (2/80)* | >0.99 |
| death & Q-wave MI | | | |
| Emergent CABG without | 0 | 0 | — |
| death or MI | | | |
| TL-CABG without death, MI, | 0 | 0 | — |
| or emergent CABG | | | |
| TL-PTCA without death, MI, | 0 | 1.3% (1/80)** | >0.99 |
| emergent CABG or | | | |
| TL-CABG | | | |

TABLE 14
MACE at six months (non-hierarchical)

| | Control BMS | BIOLIMUS A9 | p-Value |
|---|---|---|---|
| MACE (death, MI, or TLR) | 2.5% (1/40) | 3.8% (3/80) | 0.68 |
| Death | 0 | 0 | — |
| Myocardial Infarction (Q | 2.5% (1/40) | 2.5% (2/80) | 0.40 |
| wave and | | | |
| Q wave | 0 | 0 | — |
| Non-Q wave | 2.5% (1/40) | 2.5% (2/80) | 0.40 |
| Emergent CABG | 0 | 0 | — |
| Target lesion revascularization | 0 | 1.3% (1/80) | >0.99 |
| TL-CABG | 0 | 0 | — |
| TL-PTCA | 0 | 1.3% (1/80) | >0.99 |

TABLE 15
MACE at six months (hierarchical)

| | Control BMS | BIOLIMUS A9 | p-Value |
|---|---|---|---|
| MACE (death, MI, or TLR) | 2.5% (1/40) | 3.8% (3/80) | 0.68 |
| Death | 0 | 0 | — |
| Q-wave MI without death | 0 | 0 | — |
| Non-Q wave MI without | 2.5% (1/40) | 2.5% (2/80) | 0.40 |
| death & Q-wave MI | | | |
| Emergent CABG without | 0 | 0 | — |
| death or MI | | | |
| TL-CABG without death, MI, | 0 | 0 | — |
| or emergent CABG | | | |
| TL-PTCA without death, MI, | 0 | 1.3% (1/80) | >0.99 |
| emergent CABG or | | | |
| TL-CABG | | | |

Intravascular ultrasound (IVUS) procedures performed at six-months following stent implantation revealed a neointimal volume index of 1.90% for the BMS in contrast to a 0.20% neointimal volume index in the DES group (p-value=<0.001). This represents an 89% reduction in neointimal volume in the DES group compared to the BMS control group. Similarly, the percentage of neointimal volume was recorded at 23.50% in the BMS group and 2.60% in the DES group, demonstrating a difference of 89% between the two groups.

BIOLIMUS A9 blood concentrations were analyzed in sixty-our patients. The data indicated peak blood levels of 3 ng/mL which is 20-fold lower level than therapeutic levels achieved with sirolimus or everolimus administered orally following organ transplant. The ability to achieve a therapeutic benefit with lower blood levels of a drug underscores the advantages of using BIOLIMUS A9.

Based on the clinical evaluations performed six months following the stent implantation procedure, it is apparent that both the DES and BMS groups generally had positive clinical outcomes with minimal occurrence of MACE (3.8% vs. 2.5%, respectively (P≧0.99))) and favorable pharmacokinetic (PK) data. Nonetheless, the BIOMATRIX® DES stent produced reduced neointimal hyperplasia compared to the BMS control stent. The incidence of late acquired incomplete stent apposition was low (3% in both groups), which compares favorably and is typically lower than current commercial DES.

The rates of restenosis and in-stent percent diameter stenosis in both the DES and BMS groups were lower than the rates reported using other BMS in similar bare stent trials. However, the BIOLIMUS A9-eluting DES produced a lower in-stent restenosis rate than the BMS (3.9% vs. 7.7%, respectively; p=0.4) and decreased late loss (0.26 mm vs. 0.74 mm, respectively; p=<0.001). As noted above IVUS data indicated a reduced percentage of neointimal volume (2.6% vs. 23.5%, p-value=<0.001) in the DES group compared to the BMS control group. No restenosis or sub-acute thrombosis was noted.

The results obtained from the STEALTH study are compared to those obtained in other DES studies, as seen in Table 16 below:

TABLE 16
Comparison of DES Clinical Results

| | Measures of Safety | | | Measures of Efficacy | | |
|---|---|---|---|---|---|---|
| | | Event- | | In-stent | | |
| | | free | | binary | In-stent | In stent % |
| | MACE | Survival | TVR | restenosis | late loss | diameter |
| DES Trial | (%) | (%) | (%) | (%) | (mm) | stenosis |
| BIOMATRIX ®/BIOLIMUS | 3.8¹ | 96.3¹ | 1.3¹ | 2.6¹ | 0.19¹ | 20.8¹ |
| A9 (STEALTH) | | | | | | |
| CYPHER ®/sirolimus | 10.9³ | 91.0³ | 7.9² | 3.2² | 0.17² | 23.9² |
| (SIRIUS) | | | | | | |
| CYPHER ®/sirolimus (e- | 8.0² | 91.9² | ~8.0² | 3.9² | 0.18² | |
| SIRIUS) | | | | | | |
| CHAMPION/everolimus | 6.4¹ | 93.0¹ | 3.8¹ | 0.0¹ | 0.12¹ | |
| (FUTURE I/II) | | | | | | |
| ENDEAVOR ®/zotarolimus | 2.0⁴ | 98.0⁴ | | 2.1⁴ | 0.33⁴ | 21.7⁴ |
| (Endeavor 1) | | | | | | |
| TAXUS ®/paclitaxel | 8.5³ | 91.0³ | 4.7³ | 9.1³ | 0.39³ | 26.3³ |
| (TAXUS IV) | | | | | | |
| TAXUS ®/paclitaxel (TAXUS | 16.4³ | 83.0³ | 9.1³ | 9.1³ | 0.39³ | |
| VI) | | | | | | |

Follow-up intervals:
¹6 months,
²8 months,
³9 months,
⁴4 months

Clinical results obtained 12 months following stent implantation demonstrated similar safety and efficacy for the BIOLIMUS A9 eluting DES compared to the BMS, as shown in Table 17. Note that the death events (*) were non-cardiac in nature, including one case of diabetic foot syndrome in the BMS group and one case of acute leukemia in the DMS group.

TABLE 17
STEALTH results at 12 months

| | 6 Months | | Late (12+) Months | |
|---|---|---|---|---|
| | S-Stent ™ | BIOMATRIX ® | S-Stent ™ | BIOMATRIX ® |
| MACE | 2.5% | 3.8% | 5.0% | 5.1% |
| Death* | 0.0% | 0.0% | 2.5% | 1.3% |
| Q Wave | 0.0% | 1.3% | 0.0% | 1.3% |
| MI | | | | |
| Non-Q | 2.5% | 1.3% | 2.5% | 1.3% |
| Wave MI | | | | |
| TLR- | 0.0% | 0.0% | 0.0% | 0.0% |
| CABG | | | | |
| TLR- | 0.0% | 1.3% | 0.0% | 1.3% |
| PTCA | | | | |

Based on the results of the six and twelve-month clinical evaluations in the STEALTH randomized trial, it was apparent that the BIOLIMUS A9 eluting DES achieved the primary endpoint of non-inferiority for 6-month in-segment late loss, but showed superior efficacy for both in-segment (0.09±0.31 for DES vs. 0.48±0.43 for BMS, p<0.001) and in-stent (0.19±39 for DES vs 0.76±0.45 for BMS, p<0.001) late loss compared with the BMS at 6 months. This benefit was achieved without a significant increase in adverse safety outcomes as determined by MACE in the first 30 days (3.8% for DES vs. 2.5% for BMS) or at 12 months (5.1 % for DES vs. 5.0% for BMS) compared with the BMS. There was one incidence of acute (<30 days) stent thrombosis in the DES group and no subacute or late stent thromboses in either group.

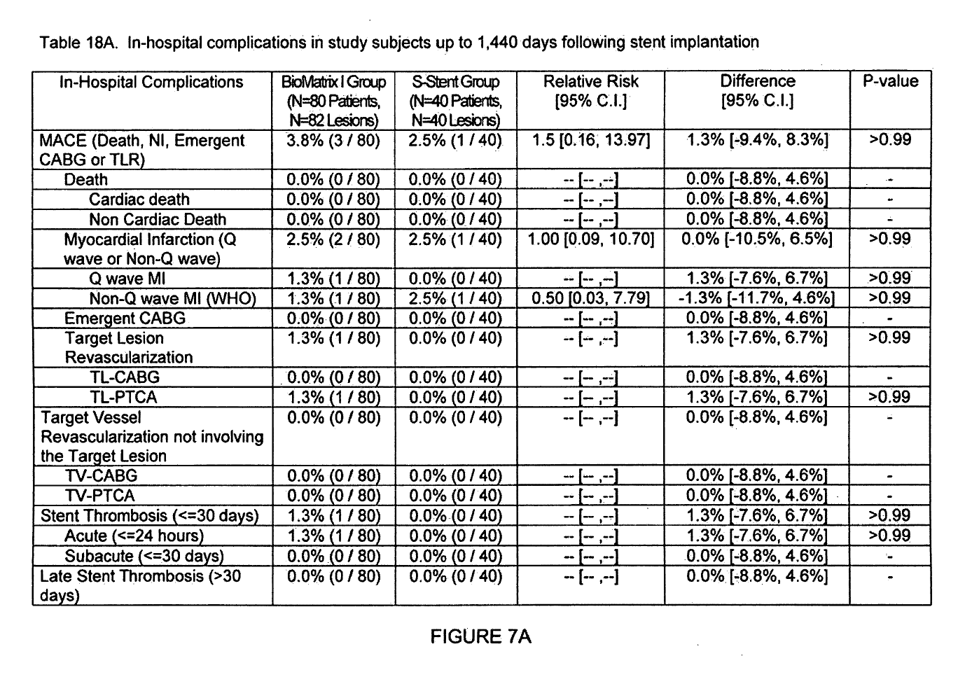

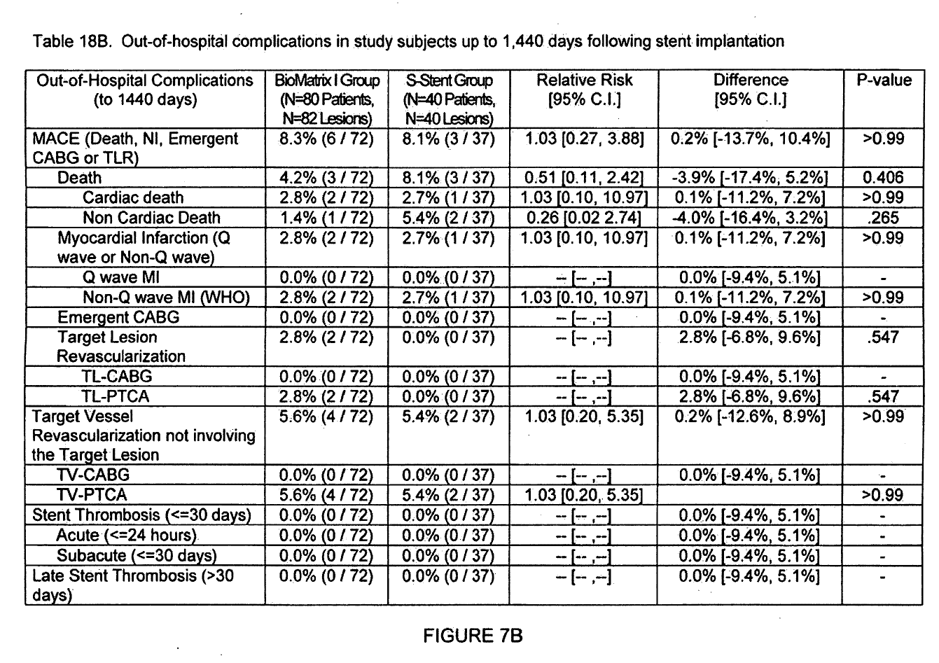

Additional data obtained from the clinical evaluation of stent recipients for up to about 4 years are summarized in Tables 18A and 18B (FIGS. 7A and 7B). The confidence intervals (CI), based on the Wilson method, are shown in brackets below. P-values were based on Fisher's exact test. Patients included in the denominator are those having at least 1,380 days of follow-up or having a MACE event.

As shown in Tables 18A and 18B, the event-free survival from MACE at 720 days was 93.7% for the DES group and 92.5% for the BMS group, with a difference of 1.2% (p-value=0.817). The event free survival from MACE at 1,080 days was 91.1% for the DES group and 92.5% for the BMS group, with a difference of −1.4% (p-value=0.803). The event free survival from MACE at 1,440 days was 89.7% for the DES group and 90.0% for the BMS group, with a difference of −0.3% (p-value=0.970). All values were determined using a 95% confidence interval.

The results of the STEALTH randomized clinical trial demonstrated that a limus-eluting DES, in particular a BIOLIMUS A9 eluting BIOMATRIX® DES, achieved the primary endpoint of non-inferiority for 6-month in-segment late loss compared with a control BMS (i.e., the S-Stent™) in the patient population defined, e.g., in Table 7. The BIOMATRIX® DES was also statistically superior to the BMS in terms of both in-segment (0.09±0.31 mm vs. 0.48±0.43 mm, respectively, p<0.001 as shown in the 12 month report) and in-stent (0.19±39 mm vs. 0.76±0.45 mm, respectively, p<0.001) late loss at 6 months.

This benefit in therapeutic efficiency was achieved without an adverse effect in safety, as assessed by MACE in the first 30, 90, or even 180 days (3.8% vs. 2.5%, for the DES and BMS group, respectively, at each time point). The safety outcomes at 360 days (5.1% vs. 5.0%, respectively), 720 days (6.5% vs. 7.5%, respectively), 1,080 days (9.2% vs. 7.5%, respectively) and 1440 days (11.0% vs. 10.8%, respectively) were all within acceptable ranges.

These results demonstrate that a metal stent with a limus/polymer coating achieves reduced very late restenosis rates in human subjects over about a four year period (i.e., 1,440 days) following stent implantation, and that patients remain free of stent thrombosis between about one month (i.e., 30 days) and at least about four years (i.e., 1,440 days) following stent implantation.

C. Pharmacokinetic Data

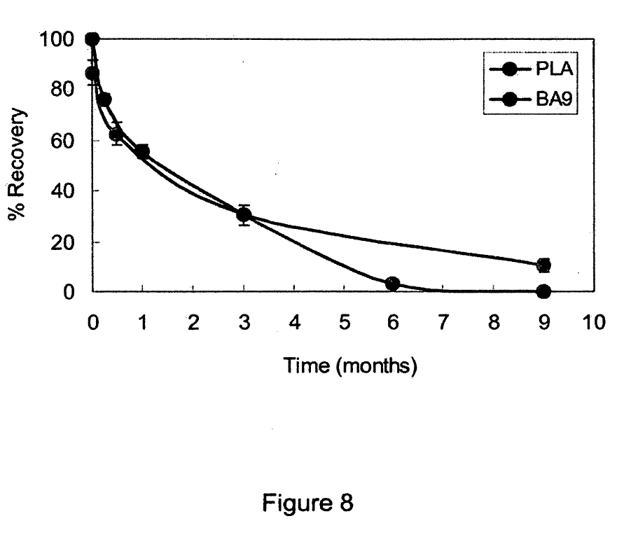

Limus drug release and polymer degradation were monitored following stent implantation in coronary vessels of pigs. The results are shown in the graph in FIG. 8. BIOLIMUS A9 release from BIOMATRIX® DES stents was approximately concurrent with PLA polymer degradation. Drug release and polymer degradation are nearly complete at 9 months. By one year there was no detectable PLA or drug in the vessel wall or in surrounding tissues.

The complete release/degradation of drug and polymer promote improved healing and reduced inflammation compared to conventional stents, wherein a polymer permanently encapsulates the stent and continues to release at least small amounts of an antirestenosis drug for much longer than one year. The concurrent release/degradation of drug and polymer means that the immune systems of subjects are not exposed to polymer in the absence of a therapeutic agent, reducing unwanted immunological and foreign body reactions.

### EXAMPLES

The following examples relate to particular procedures used in studies described, herein, and should in no way be considered limiting.

### Example 1

### Stent Implantation Procedure #1

The following procedure was used for implantation of the BIOMATRIX® DES:

### Step 1:

A 75 unit/kg loading dose of intravenous heparin was administered and an activated clotting time (ACT) level >250 seconds was maintained for the remainder of the procedure. ACT measurements were taken at least once every 60 minutes.

### Step 2:

300 mg loading dose of PLAVIX® and 300 mg loading dose of aspirin was administered to the subject who was not already on aspirin. TICLID® (500 mg loading dose and 250 mg b.i.d. for 2 weeks) was also used in some cases instead of PLAVIX®. In cases where a Factor IIb/IIIa antagonist was administered, the ACT was maintained between 225 and 300 seconds.

### Step 3:

Selected coronary artery lesion(s) was/were predilated with a balloon that was at least 4 mm shorter than the length of the stent implanted. Only conventional balloon angioplasty was used prior to stent implant. A brief cine film was recorded during the procedure to demonstrate the treatment position. Pre-dilated areas were covered completely with the DES or control BMS stent. Post-dilatations were optional and were only done using short balloons and within the boundaries of the implanted stent.

### Step 4:

Angiographic images on CDR documented two orthogonal projections in the pre-procedure angiogram following intra-coronary nitroglycerin injection. A frame of the guide catheter filled with contrast was included in each case. The CRF was used to determine, for example: (i) reference vessel diameter just proximal and distal to the lesion, (ii) minimum lumen diameter, (iii) lesion length, (iv) diameter stenosis, (v) TIMI flow, and (vi) dissection grade if a dissection was noted.

### Example 2

### Stent Implantation Procedure #2 (Interventional Procedure)

The following procedure was used for interventional stent implantation. Where possible, a single stent was implanted to provide complete lengthwise coverage of a lesion; however, personnel performing the procedures had discretion to implant a second, overlapping stent in the event of edge dissection or placement error.

### Step 1:

The BIOMATRlX® DES was advanced to the lesion site using the device illustrated in FIG. 5 and the balloon was expanded to implant the stent according to the deployment balloon/pressure expansion table in the stent package insert to obtain a final dilation balloon diameter of between 105%-110% of reference vessel diameter, and a residual diameter stenosis of <20%.

### Step 2:

Post dilation using a non-compliant balloon was used only in cases where the initial result of stent deployment did not achieve the above specified post-deployment stent diameter along the entire length of the stent.

### Step 2:

The final angiographic result (post-intervention) was documented on optical media following intra-coronary nitroglycerin injection in the same two orthogonal projections as the pre-procedure angiogram described above. The angiogram included a frame of the guide catheter filled with contrast. The following angiographic measurements were recorded on the case report form (CRF): (i) reference vessel diameter just proximal and distal to the lesion, (ii) minimum lumen diameter, (iii) lesion length, (iv) diameter stenosis, and (v) TIMI flow.

### Example 3

### Preparation of 42-O-(2-ethoxylethyl)-rapamycin (BIOLIMUS-A9)

A. Synthesis of 2-Ethoxyethanol Triflate

To a stirred cooled (0° C.) solution containing 4.28 g 2-ethoxyethanol (Aldrich Chemical) and 10.14 g 2,6-lutidine in 160 mL CH₂Cl₂, 19.74 g trifluoromethanesulfonic (triflic) anhydride was slowly added under nitrogen. The mixture was washed with four portions of 200 mL brine and the organic solution dried over anhydrous sodium sulfate filtered and concentrated. The residue was purified by flash chromatography on silica gel, 200-400 mesh (75:25 hexanes-ethyl ether (v/v)) to produce the triflate of 2-ethoxyethanol: light yellow liquid, TLC R_{f}=0.47 using hexanes-ethyl ether 75:25 (v/v).

B. Reaction of 2-Ethoethanol Triflate with Rapamycin

To a stirred solution containing 1 g rapamycin and 7.66 g 2,6-lutidine in 14.65 mL toluene held at 60° C. was added 5.81 g 2-ethoxyethanol triflate. Stirring was continued for 90 minutes after which 50 mL ethyl acetate was added to the reaction and the solution was washed with 50 mL 1 M HCl. The organic material was washed with distilled deionized water until the pH of wash solution was neutral. The organic solution was dried over anhydrous sodium sulfate, filtered, and concentrated. The residue was purified by flash chromatography on silica gel 200-400 mesh (40:60 hexane-ethyl acetate (v/v)) to produce 210 mg 42-O-(2-ethoxyethyl) rapamycinl: TLC R_{f}=0.41 using 40:60 hexane-ethyl acetate (v/v). MS (ESI) m/z 1008.5 C₅₅H₈₇NNaO₁₄.

The chemical structure of 42-O-(2ethoxyethyl)rapamycin was further verified by mass spectrometric tandem quadrupole experiments (CAD experiments, collisionally activated dissociation). These studies were performed on a Thermo Finnigan, LCQ Advantage quadrupole ion trap mass spectrometer equipped with an electrospray ionization source. Direct infusion of the sample in methanol was done at a flow rate of 2.5 μL/min from a syringe. CAD experiments were carried out after obtaining maximum signal intensity. Helium was used as the collision gas. Collision energy was tuned during the MS/MS experiments to obtain the full range of fragments. The fragmentation patterns indicated the presence of the ion pair 1008.5→417.5. These results are consistent with the chemical structure of 42-O-(2ethoxyethyl)rapamycin.

Purity of the product was determined by HPLC. A Zorbax SB-C18 HPCL system was used, with a 4.6 mm ID×250 mm (5 μm) column. A step gradient solvent system was utilized consisting of 100% (10% methanol-water), one minute, 50% (10% methanol-water)/50% methanol, one minute; 25% (10% methanol-water)/75% methanol, one minute; 100% methanol. A flow rate of 1.0 mL was used. Column temperature was 55° C. 2.0 μg of 42-O-(2-ethoxyethyl)rapamycin was injected onto the column in a volume of 20 μL methanol. Detection by UV at 278 nm. Purity was 98.7% (average of three runs; SD=0.2).

What is claimed is:

1. A method of stent-placement percutaneous coronary intervention (STPCI) that is effective to achieve a significant reduction in the extent of late restenosis, relative to that observed in a STPCI method in which a bare metal stent is placed at the site of vessel occlusion, without a concomitant increase in stent thrombosis up to 4 years following stent placement, comprising
selecting as a subject for the method, an individual having a coronary occlusion condition characterized by a baseline stenosis >50%, a left main occlusion <50%, and a left ventricular ejection fraction (LVEF) of at least 30%, and
implanting at the site of the vessel occlusion, a drug-eluting stent having a metal body whose outer surfaces have been treated to enhance the adhesion of a biodegradable polymer coating and having on the treated outer surfaces, a drug/polymer coating formulated to contain at least 40% by weight of a macrocyclic triene anti-restenosis drug in a polylactic acid (PLA) polymer, at a coating thickness such that the both the drug and coating are completely released from the stent body over a period of between 6-12 months.

2. The method of claim 1, wherein the drug-polymer coating contains a 1:1 mixture by weight of drug and polymer, and the PLA polymer is D,L-PLA.

3. The method of claim 1, wherein the limus drug is selected from the group consisting of rapamycin, everolimus, zotarolimus, biolimus A9, novolimus, RAPALOG #AP23573.

4. The method of claim 3, wherein the limus drug is biolimus A9.

5. The method of claim 1, wherein the coating is applied in an amount equal to about 14-16 μg/mm stent length.

6. The method of claim 1, wherein the stent body outer surfaces have been treated by applying a coating of parylene to the surfaces by vapor deposition.

7. The method of claim 1, wherein the stent body outer surfaces have been treated by plasma cleaning.

8. The method of claim 1, wherein the matrix is coated on the exterior surfaces of the stent in an amount of about 15.6 μg/mm of stent length.

9. The method of claim 1, wherein the biodegradable polymer is polylactic acid (PLA).

10. The method of claim 9, wherein the biodegradable polymer is D,L PLA.
